(19) United States
(12) Patent Application Publication
(10) Pub. No.: US 2021/0015701 A1
(43) Pub. Date: Jan. 21, 2021

(54) SYSTEMS AND METHODS FOR PROVIDING A MENSTRUAL CRAMP RELIEF BELT

(71) Applicant: SMS Products LLC, Riverton, UT (US)

(72) Inventors: Megan Nuffer, Riverton, UT (US); Shane Brady, Riverton, UT (US)

(73) Assignee: SMS Products LLC, Riverton, UT (US)

(21) Appl. No.: 16/930,072

(22) Filed: Jul. 15, 2020

Related U.S. Application Data
(60) Provisional application No. 62/874,905, filed on Jul. 16, 2019.

(51) Int. Cl. A61H 23/02

(52) U.S. Cl. CPC A61H 23/02; A61H 2201/165; A61H 2201/1628; A61N 1/36021; A61H 2201/1623; A61H 2201/10; A61H 2201/0214; A61H 2201/0207

(57) ABSTRACT

An abdominal belt that is configured to apply heat, vibration, and/or pressure to a lower abdominal portion of a user's body to alleviate pain and discomfort (e.g., from PMS) is disclosed. In some cases, the abdominal belt has a first end, a second end, and an abdominal pad disposed at the belt's second end. In some cases, the abdominal pad includes one or more heating elements and/or vibrating mechanisms. Indeed, in some cases, the pad includes two vibrating mechanisms configured to be disposed over a user's ovaries when the belt is worn. In some cases, the abdominal pad also defines an opening through which the belt's first end can extend. Additionally, the belt includes a coupling mechanism that couples the first end to a middle portion of the belt when the belt is extended through the opening and the first end of the belt is folded back on the middle portion. Other implementations are described.

### RELATED APPLICATIONS

This patent application claims priority to U.S. Provisional Patent Application Ser. No. 62/874,905 (Attorney Docket No. 30168.2), filed Jul. 16, 2019, and entitled SYSTEMS AND METHODS FOR PROVIDING A MENSTRUAL CRAMP RELIEF BELT; the entire disclosure of which is hereby incorporated by reference.

### BACKGROUND OF THE INVENTION

### Field of the Invention

The present invention relates to menstrual cramps, premenstrual syndrome (PMS), ovarian swelling, and abdominal pain. More particularly, some implementations of the described invention relate to systems and methods for applying heat, vibration, and/or pressure to a lower abdominal portion of a user's body in an effort to help alleviate pain and discomfort associated with menstrual cramping, PMS, ovarian swelling, menopause, and/or a variety of other abdominal pains. While the described systems can include any suitable component, in some cases, they include a belt having a first end, a second end, and an abdominal pad that is disposed at the second end of the belt. In some cases, the abdominal pad includes one or more heating elements and/or vibrating mechanisms. Indeed, in some cases, the pad includes two vibrating mechanisms, which are spaced apart and otherwise configured to be disposed over a user's ovaries when the belt is worn. In some cases, the abdominal pad also defines an opening through which the first end of the belt is configured to extend, and the belt includes a coupling mechanism that is configured to couple the first end of the belt to a middle portion of the belt when the belt is extended through the opening and when the first end of the belt is folded back on to the middle portion of the belt.

### Background and Related Art

In advance of and/or during menstruation (or a menstrual period) many people experience abdominal pain, cramping, bloating, water retention, pelvic pressure, fatigue, headaches, migraines, mood swings, irritability, lower back pain, constipation, diarrhea, sore breasts, sensitivity to light or sound, changes in sleep patterns, and/or a variety of other uncomfortable and undesirable symptoms. In an effort to reduce the symptoms associated with menstruation, people often employ a number of different strategies, with varying degrees of success. Indeed, in some cases, to ease the pains associated with menstruation, some people: drink more water in an effort to ease abdominal bloating, take one or more supplements (e.g., magnesium, vitamin B-6, vitamin D, folic acid, calcium, etc.) to reduce cramps or mood swings, take pain medications (e.g., MIDOL™, ibuprofen, aspirin, acetaminophen, caffeine, naproxen sodium, etc.) to alleviate cramping and aches, seek to get more sleep, try to get more exercise, eat a balanced diet, and/or otherwise try to avoid, shorten, or reduce such symptoms.

While many conventional methods that are used to alleviate the symptoms of menstruation can be at least marginally effective, such methods are not necessarily without their shortcomings. Indeed, despite their many efforts, some people still find it hard to reduce abdominal pain, cramping, swelling, and/or other discomforts that are associated with menstruation. Accordingly, despite the variety of possible treatments, many people are still forced to wait out their menstrual periods and the associated symptoms.

Thus, while systems and methods currently exist that are used to reduce or shorten the symptoms associated with menstruation, some challenges still exist, including those listed above. Accordingly, it would be an improvement in the art to augment or even replace current techniques with other techniques.

### SUMMARY OF THE INVENTION

The present invention relates to menstrual cramps, premenstrual syndrome (PMS), ovarian swelling, and abdominal pain. More particularly, some implementations of the described invention relate to systems and methods for applying heat, vibration, and/or pressure to a lower abdominal portion of a user's body in an effort to help alleviate pain and discomfort associated with menstrual cramping, PMS, ovarian swelling, menopause, and/or a variety of other abdominal pains. While the described systems can include any suitable component, in some cases, they include a belt having a first end, a second end, and an abdominal pad that is disposed at the second end of the belt. In some cases, the abdominal pad includes one or more heating elements and/or vibrating mechanisms. Indeed, in some cases, the pad includes two vibrating mechanisms, which are spaced apart and otherwise configured to be disposed over a user's ovaries when the belt is worn. In some cases, the abdominal pad also defines an opening through which the first end of the belt is configured to extend, and the belt includes a coupling mechanism that is configured to couple the first end of the belt to a middle portion of the belt when the belt is extended through the opening and when the first end of the belt is folded back on to the middle portion of the belt.

In some implementations, the described abdominal belt, menstrual belt, or menstrual cramp belt includes a belt that has a first end and a second end, with an abdominal pad disposed at the second end of the belt. In some such implementations, the abdominal pad includes a first vibrating mechanism, a second vibrating mechanism, and one or more heating elements. Additionally, in some such implementations, the vibrating mechanisms are coupled to the abdominal pad so as to be disposed adjacent to a user's ovaries when the user wears the belt. Indeed, in some implementations, the first and second vibrating mechanisms are disposed at substantially the same level (or vertical position) on the abdominal pad and are separated from each other on the abdominal pad (e.g., having a distance between an innermost edge of the two vibrating mechanisms) by any suitable distance that allows the vibrating mechanisms to be disposed adjacent to a user's ovaries, including, without limitation, being separated by a distance that is less than about 12 cm.

Although the abdominal pad can include any suitable material, in some cases, it includes an inner material that is configured to face a user when the user wears the menstrual belt, and an outer material that is disposed substantially opposite to the inner material. In this regard, the inner material and the outer material can comprise any suitable material or materials that allow the belt to provide heat, vibration, and/or pressure to a lower abdomen of a person wearing the belt. Some non-limiting examples of such materials include neoprene, polyester, cotton, cotton polyester blends, rayon, wool, leather, fabric, linen, canvas, and/or any other suitable material. In some cases, however, the inner material comprises polyester and/or any other suitable material, and the outer material comprises neoprene and/or any other suitable material. In this regard, some implementations of the outer material are configured to better at insulating heat, less breathable, better at absorbing vibrations, and/or less flexible than some embodiments of the inner material. In this manner, some such implementations of the outer material are configured to drive heat, vibrations, and/or pressure into (as opposed to away from) the user's abdomen, and the inner material is configured to help (or at least do little to impede) the belt from providing heat, vibrations, and/or pressure to the user's abdomen.

Although some implementations of the heating element comprise one or more wires, filaments, grids, and/or nets of heating elements that extend across the abdominal pad (and/or any other suitable portion of the abdominal belt, including, without limitation, the belt), in some implementations, the abdominal pad comprises one or more heating elements that are disposed in various locations (or nodes) across the abdominal pad and/or the belt. While such nodes can be disposed in any suitable location with respect to the abdominal pad (e.g., above, below, between, on one or more sides of, flanking, and/or in any other suitable relation to the vibrating mechanisms), in some cases, the nodes (or heating nodes) are disposed in locations on the abdominal pad that are configured to correspond to one or more nerve endings, veins, arteries, acupuncture points, organs, ovaries, uteruses, uterine tubes, and/or other similar trigger points that can increase blood flow, promote relaxation, ease bloating, reduce cramping, dampen pain, and/or otherwise reduce menstrual symptoms (and/or other abdominal pains) when heat is applied to such points. Indeed, in some implementations, one or more heat nodes are configured to be disposed over a user's uterus, ovaries, uterine tubes, abdominal muscles, bowels, and/or other such trigger points when the abdominal belt is properly worn.

Although the heating nodes can have any suitable characteristic, in some embodiments, they have a length and/or width that are less than about 6 cm (or within any subrange thereof). Indeed, in some cases, the heating nodes are less than about 1.5 cm by 1.5 cm in length and width. Moreover, although some implementations of the heat nodes are disposed within the abdominal belt and do not protrude from an inner (or front or first) surface or outer (or back or second) surface of the belt, in some other implementations, one or more of the heat nodes are configured to protrude from the inner and/or outer surface of the belt.

Additionally, in some implementations, one or more of the vibrating mechanisms are configured to protrude from an inner or first side of the abdominal pad, such that the vibrating mechanisms are configured to press into (e.g., to apply more pressure than another portion of the abdominal pad to) an abdomen of the user when the user wears the abdominal belt over the user's abdomen and the belt is tightened onto the user.

The described abdominal belt can be modified in any suitable manner. Indeed, in some implementations, in addition to having one or more vibrating mechanisms and/or heating elements on the abdominal pad, the abdominal belt further comprises one or more vibrating mechanisms and/or heating elements that are disposed at various locations on the belt.

Moreover, in some implementations, in addition to the abdominal pad, the abdominal belt comprises a back pad that comprises one or more vibrating mechanisms and/or heating elements and that is configured to provide heat, vibration, and/or pressure to the user's back.

In some implementations, instead of comprising an abdominal pad, the described systems include a back pad that comprises one or more vibrating mechanisms and/or heating elements and that is configured to provide heat, vibration, and/or pressure to the user's back (e.g., to the user's lower back).

As some additional examples of suitable modifications, in some implementations, the described abdominal belt includes one or more transcutaneous electrical nerve stimulation devices, kneading rollers, percussion heads, massage beads, cooling elements, misters, steamers, aroma therapy diffusers, programs that control the heating elements and/or the vibration mechanisms, bladders that are configured to inflate to provide more pressure to a user, mechanisms that are configured to automatically tighten and/or loosen the belt on a user, and/or any other suitable component or characteristic that still allows the belt to be used to provide heat, vibration, and/or pressure to the user.

While the described abdominal belt can be used in any suitable manner, in some implementations, a method for using belt includes a method for applying at least one of heat, vibration, and pressure to an abdominal portion of a user. In some such cases, the method includes obtaining a belt having a first end, a second end, and an abdominal pad comprising a first vibrating mechanism, a second vibrating element, and a first heating element, wherein a first end portion of the abdominal pad is coupled to the second end of the belt, and wherein the first and second vibrating mechanisms are separated from each other on the abdominal pad by less than about 12 cm. In some such cases, the method further includes coupling the belt on the user such that the abdominal pad is disposed over a lower abdomen of the user and such that the first and second vibrating mechanisms are respectively disposed adjacent to a first and a second ovary of the user.

While the described methods and processes can be particularly useful for treating abdominal pain, bloating, swelling, cramping, and/or other abdominal symptoms related to menstruation, those skilled in the art will appreciate that the described systems and methods can be used in a variety of different applications and in a variety of different areas of manufacture. For instance, the described systems and methods can be used not only for use prior to and/or during menstruation, but some implementations of the described systems and methods are configured to be used in any other suitable location and/or application. Indeed, in some implementations, the described abdominal belt is configured to be used to treat abdominal pains and maladies that are associated with or result from constipation, gas, diarrhea, bowel blockages, bloating, and/or a variety of other abdominal conditions. Additionally, in some implementations, the described systems and methods are used to provide heat, vibration, and/or pressure to a user's lower back.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above-recited and other features and advantages of the present invention are obtained, a more particular description of the described inventions will be rendered by reference to specific embodiments thereof, which are illustrated in the appended drawings. Understanding that the drawings are not necessarily drawn to scale or in proper proportion, and that the drawings depict only typical embodiments of the present inventions and are not, therefore, to be considered as limiting the scope of the inventions, the present inventions will be described and explained with additional specificity and detail through the use of the accompanying drawings in which:

FIG. 1 illustrates a front perspective view of a user wearing an abdominal belt in accordance with a representative embodiment;

FIGS. 2A-2B each illustrate a plan view of an outer (or second) surface of the abdominal belt in accordance with a representative embodiment;

FIG. 2C illustrates a plan view of an inner surface of the abdominal belt in accordance with a representative embodiment;

FIG. 2D illustrates a view of an outer surface of an abdominal pad of the abdominal belt in accordance with a representative embodiment;

FIG. 2E-2K each illustrate a different view of the abdominal belt in accordance with some embodiments;

FIG. 3 illustrates a view of a female body, identifying potential pressure points that may benefit from the application of heat and/or vibration during menstruation and/or PMS; in such figure a view of a representative embodiment of the abdominal belt and some of its components is also shown;

FIG. 4 illustrates a view of a female body, identifying potential pressure points that may benefit from the application of heat and/or vibration during menstruation and/or PMS;

FIG. 5A-5M illustrate various views of a back belt in accordance with a representative embodiment;

FIG. 6 illustrates a diagram of a representative operating environment in accordance with some embodiments of the described belts; and

FIG. 7 illustrates a diagram showing a representative networked environment for use with some embodiments of the described belts.

### DETAILED DESCRIPTION OF THE INVENTION

The present invention relates to menstrual cramps, premenstrual syndrome (PMS), ovarian swelling, and abdominal pain. More particularly, some implementations of the described invention relate to systems and methods for applying heat, vibration, and/or pressure to a lower abdominal portion of a user's body in an effort to help alleviate pain and discomfort associated with menstrual cramping, PMS, ovarian swelling, menopause, and/or a variety of other abdominal pains. While the described systems can include any suitable component, in some cases, they include a belt having a first end, a second end, and an abdominal pad that is disposed at the second end of the belt. In some cases, the abdominal pad includes one or more heating elements and/or vibrating mechanisms. Indeed, in some cases, the pad includes two vibrating mechanisms, which are spaced apart and otherwise configured to be disposed over a user's ovaries when the belt is worn. In some cases, the abdominal pad also defines an opening through which the first end of the belt is configured to extend, and the belt includes a coupling mechanism that is configured to couple the first end of the belt to a middle portion of the belt when the belt is extended through the opening and when the first end of the belt is folded back on to the middle portion of the belt.

The following disclosure of the present invention is grouped into two subheadings, namely “Abdominal Belt” and “Representative Operating Environment.” The utilization of the subheadings is for convenience of the reader only and is not to be construed as being limiting in any sense.

### Abdominal Belt

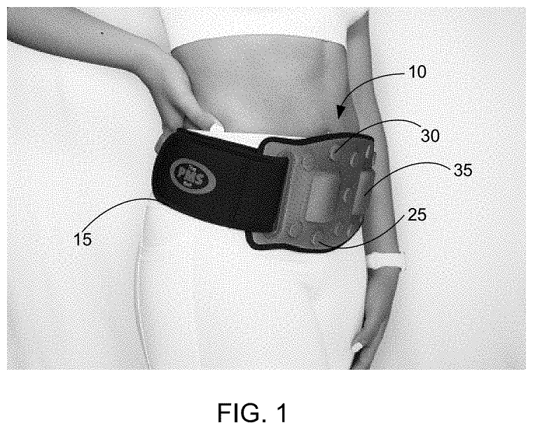

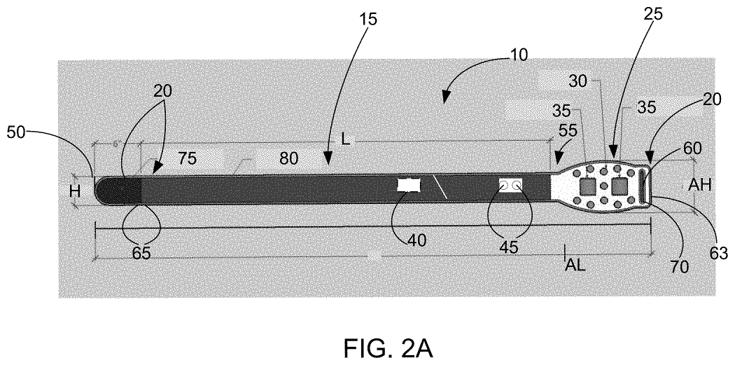

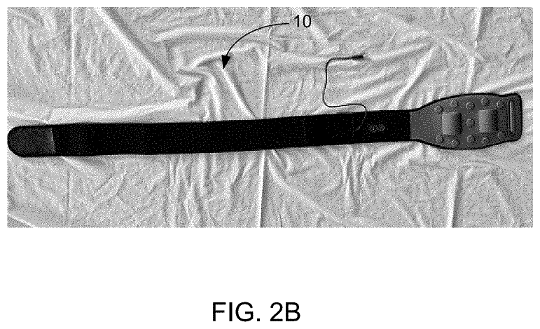

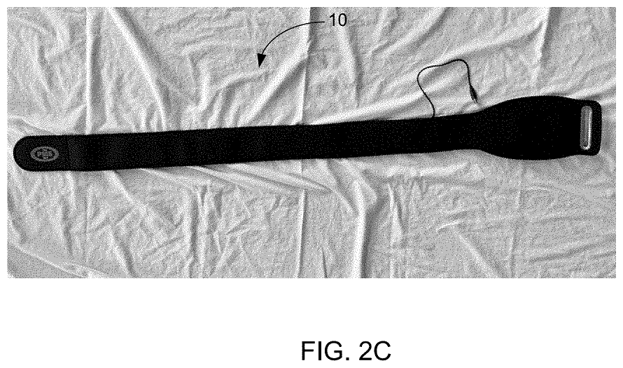

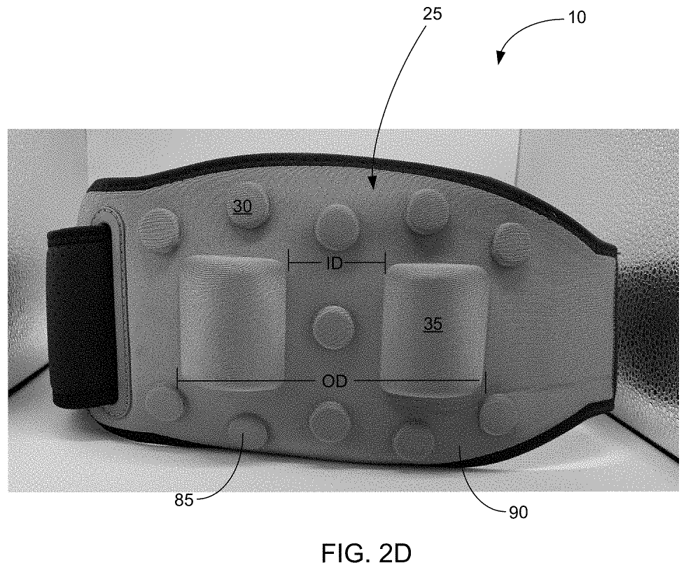

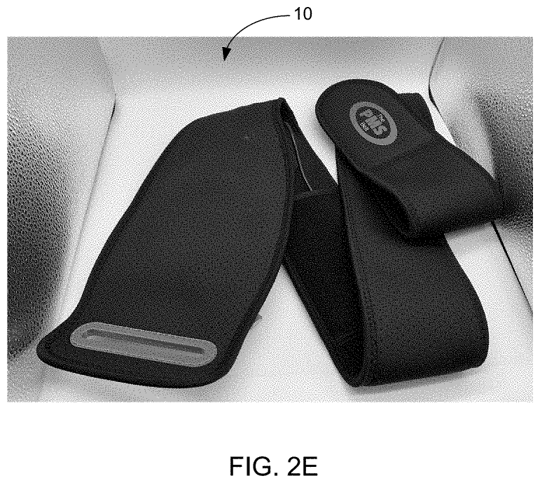

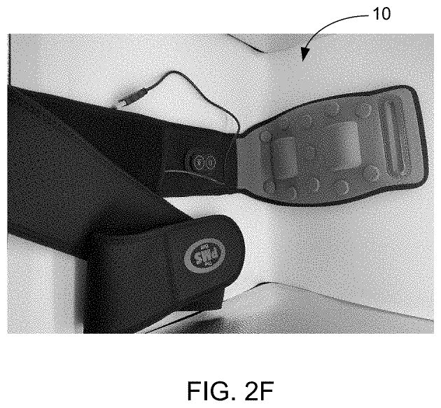

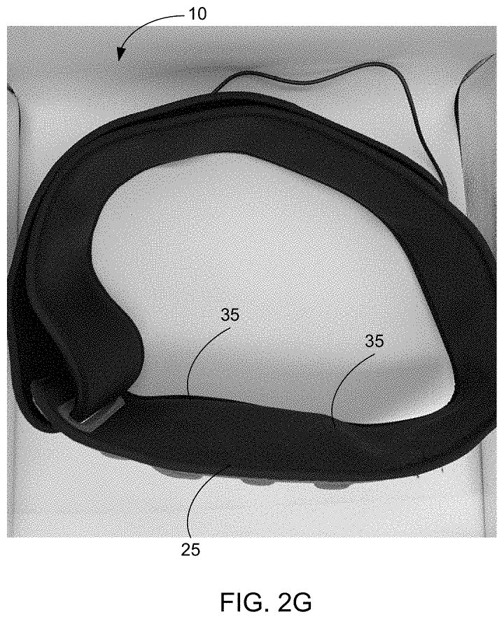

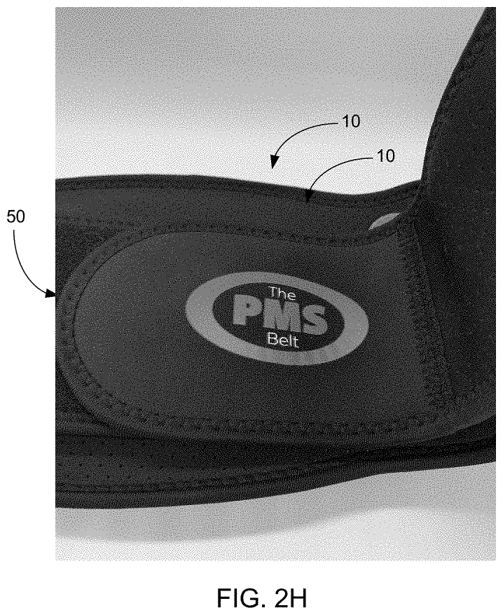

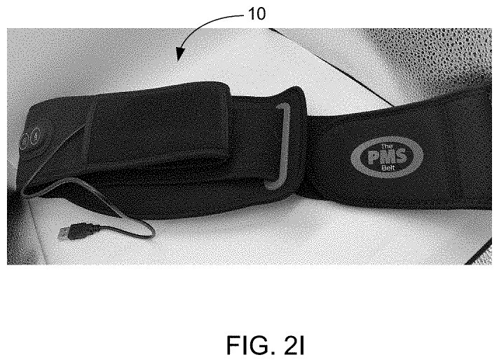

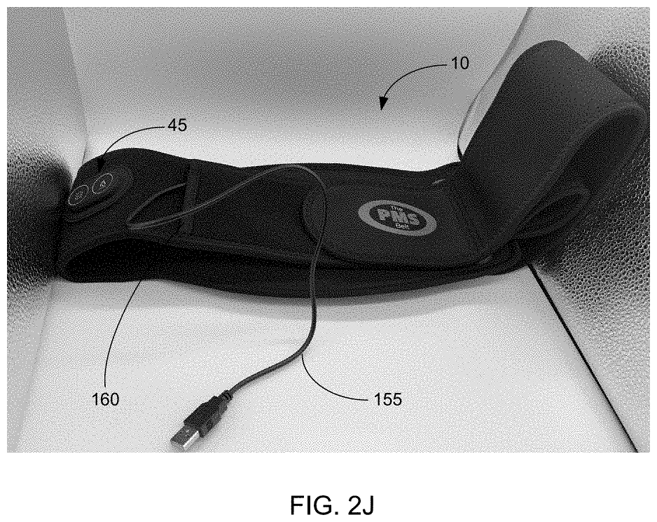

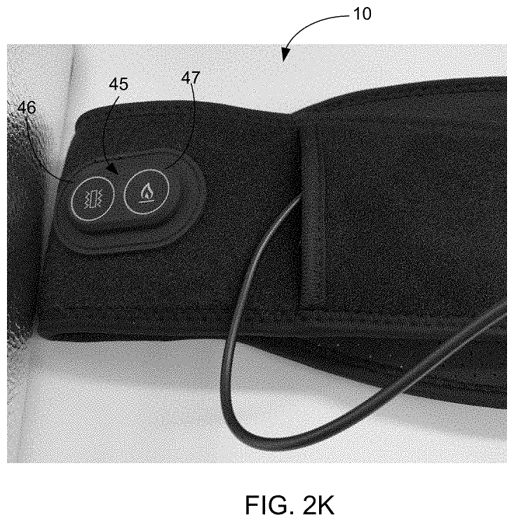

As mentioned, the described systems and methods relate to an abdominal belt that is configured to provide heat, vibration, and/or pressure to a lower abdominal portion (namely a front lower abdominal portion) of a user. In some embodiments, the abdominal belt is configured to provide heat, vibration, and/or pressure to a variety of specific places on a user's abdomen, such as to the user's ovaries, uterus, and/or uterine tubes. In this regard, while the described abdominal belt can have any suitable component or characteristic that allows it to perform such functions, FIGS. 1 and 2A show some representative embodiments in which the described abdominal belt 10 comprises one or more belts (or belt portions) 15, coupling mechanisms 20, abdominal pads 25, heating elements 30, vibrating mechanisms 35, power supplies (or sources) 40, controls 45, and/or other suitable components.

With respect to the belt (or belt portions) 15, the belt portion of the abdominal belt 10 can comprise any suitable characteristic or component that allows it to hold the abdominal pad 25 over a user's abdomen (e.g., as shown in FIG. 1) to provide heat, vibration, and/or pressure to one or more portions of the abdomen. In this regard, the belt can be any suitable length that allows it to function as described herein. For instance, in some embodiments, the belt has a length L (as shown in FIG. 2A) between its first end 50 and second end 55 (or the portion of the belt that extends from and/or is coupled to the abdominal pad 25) of between about 0.5 m and about 3 m (or within any subrange thereof). Indeed, in some embodiments, length of the belt is between about 0.8 m and about 1.8 m (e.g., about 1.2 m±0.2 m). Thus, some embodiments of the belt are configured to be used by users of a wide variety of sizes.

The belt 15 can be any suitable height H (as shown in FIG. 2A) that allows the belt to function as intended. In some embodiments, the belt has a height (e.g., a distance between its upper and lower sides) of between about 1.27 cm and about 30.5 cm (or within any subrange thereof). Indeed, in some embodiments, the belt has a height between about 5 cm and about 15 cm (e.g., about 9.5 cm±2 cm). In some cases in which the belt is relatively short, the belt can be wrapped around the user to hold the abdominal pad 25 in place while not unduly restricting the waist and/or back of the user. Thus, some embodiments of the abdominal belt 10 are able to provide desired heat, vibration, and/or pressure to the abdomen, while being comfortable around the user's waist and back.

The belt 15 can also be any suitable shape that allows the abdominal belt 10 to function as intended. Some non-limiting examples of suitable shapes include having one or more portions that are substantially straight, bowed, wavy, curved, shaped to ergonomically fit one or more specific portions of a user's body, symmetrical, asymmetrical, and/or any other suitable shape. By way of non-limiting illustration, FIG. 2A shows an embodiment in which the belt 15 is substantially straight between its first 50 and second 55 ends.

With respect to the coupling mechanism 20, the abdominal belt 10 can comprise any suitable mechanism that is configured to selectively bind the abdominal belt to a user's abdomen and to release the belt therefrom. Some examples of suitable mechanisms include, but are not limited to, one or more, belt buckles, screw closure buckles, clamp closure buckles, stitch closure buckles, double sided buckles, reversible buckles, double ring buckles, auto-grip buckles, military buckles, clip buckles, auto lock buckles, grip auto lock buckles, auto press buckles, pin buckles, flip closure buckles, single tongue buckles, double tongue buckles, triple tongue buckles, wrestling buckles, automatic locking splice buckles, military utility buckles, automatic latch buckles, webbing buckles, clip on buckles, clip latch buckles, rigger buckles, snaps, buttons, hooks and eyelets and/or catches, knots, friction couplings, mechanical couplings, hook and loop fasteners, carabineers, clasps, and/or any other mechanism that is capable of selectively tightening the abdominal belt on to a user. By way of non-limiting illustration, FIG. 2A shows that in some embodiments, the coupling mechanism 20 comprises one or more openings 65 and one or more couplers 65 that are configured to allow the abdominal belt 10 to be selectively tightened to one or more of a variety of positions.

Where the abdominal belt 10 defines one or more openings 60 that are configured to receive the first end 50 (and/or any other suitable portion of the belt 15), the opening can be defined in any suitable portion of the abdominal belt, including, without limitation, between the first and second 55 ends of the belt, in any suitable portion of the abdominal pad 25, and/or in any suitable ring, loop, strap, and/or other object that is coupled to any suitable portion of the abdominal belt. By way of non-limiting illustration, FIG. 2A shows an embodiment in which the abdominal pad 25 defines the opening 60.

Where the opening 60 is defined in the abdominal belt 10 (e.g., in the abdominal pad 25 and/or in any other suitable portion), the opening can have any suitable characteristic that allows the first end 50 of the belt portion 15 to be inserted through the opening. Indeed, in some embodiments, the opening is defined by a portion of the abdominal belt; by a reinforced portion of the abdominal belt; by one or more grommets, rings, straps, carabineers, and/or other objects that are coupled to the abdominal belt; and/or by any other suitable objects. By way of non-limiting illustration, FIG. 2A shows an embodiment in which the opening 60 is defined by an elongated grommet 63 that is disposed at an end of the abdominal pad 25.

In accordance with some embodiments, after the first end 50 of the belt 15 has been extended through the opening 60, the first end (and/or any other suitable portion of the belt) is configured to be coupled to another portion of the abdominal belt 10, including, without limitation, to a middle portion of the belt (or a portion between the belt's first and second 55 ends). In this regard, the first end of the belt can couple to the middle portion (and/or any other suitable portion of the belt) in any suitable manner. In some embodiments, however, the first end of the belt is configured to couple to the middle portion of the belt via any suitable number of couplers 65 (as shown in FIG. 2A), which can include one or more hook and loop fasteners, buttons, snaps, clasps, hooks, eyelets, mechanical engagements, frictional engagements, and/or any other suitable mechanisms that are configured to allow the first end of the belt to fold back on and/or to couple to the middle portion of the belt. By way of non-limiting illustration, FIG. 2A shows an embodiment in which the belt 15 comprises a hook and loop fastener (e.g., a hook fastener 75 at the first end of the belt 15 and a loop fastener 80 at the middle portion of the belt 15 (and/or vice versa)). In some such embodiments, such a coupling mechanism allows the abdominal belt to be easily and readily tightened at a wide variety of locations (indeed, almost to an infinite number of positions).

With reference to the abdominal pad 25, that pad can have any suitable characteristic that allows it (and/or other components of the abdominal belt) to provide heat, vibration, and/or pressure to a user's abdomen. In this regard, the abdominal pad 25 can have any suitable shape that allow it to be used to provide heat, vibration, and/or pressure to portion of a user's body (e.g., the user's abdomen or lower abdomen). In some embodiments, the abdominal pad is circular, oval shaped, elliptical, oblong, hexagonal, octagonal, square, rectangular, polygonal, symmetrical, asymmetrical, regular shaped, irregular shaped, and/or any other suitable shape that allows it to function as described herein. By way of non-limiting illustration, FIGS. 2A and 2D show some embodiments in which the abdominal pad 25 comprises a rounded shape (e.g., a substantially oval-shaped portion have connecting portions that extend from its ends). In some such embodiments, the rounded shape can allow it to be relatively comfortable, without having corners of the pad that unduly digging into the user when the user leans over or otherwise moves.

The abdominal pad 25 can be any suitable size that allows it to function as intended. Indeed, in accordance with some embodiments, the abdominal pad is configured to fit comfortably over a user's lower abdomen. In some embodiments, the length of the abdominal pad (e.g., as shown in FIG. 2A as AL), which can be any suitable size, is between about 5 cm and about 50 cm (or within any subrange thereof). Indeed, in some embodiments, the abdominal pad is between about 20 cm and about 40 cm (e.g., about 30 cm±8 cm) in length. In some embodiments, the height of the abdominal pad (e.g., as shown in FIG. 2A as AH), which can be any suitable size, is between about 2 cm and about 35 cm (or within any subrange thereof). Indeed, in some embodiments, the height of the abdominal pad is between about 10 cm and about 25 cm (e.g., about 17 cm±4 cm). Thus, in some embodiments, the abdominal pad has a height that is greater than a height H of the belt 15, to allow the abdominal pad to provide the desired heat, vibration, and/or pressure across a desired portion of a user's body, while allowing the belt to be relatively comfortable on the user. Moreover, in some embodiments, the abdominal pad is smaller than is a back pad on some competing devices. Indeed, if a back pad of some conventional devices were to be worn over a user's abdomen, such back pad could be too large and would be uncomfortable to wear in such a manner.

The abdominal pad 25 and/or the belt 15 can include any suitable material or materials that allow the abdominal belt 10 to function as described herein. In some cases, the abdominal pad and/or the belt each include a single material. In some other cases, the abdominal pad and/or the belt each include an inner material that is configured to face a user when the user wears the abdominal belt, and an outer material that is disposed substantially opposite to the inner material (e.g., so as to face away from the use when worn).

Where the abdominal pad 25 and/or the belt 15 comprises an inner material and an outer material, the inner material and the outer material can comprise any suitable material or materials that allow the abdominal belt 10 to provide heat, vibration, and/or pressure to a lower abdomen of a user wearing the belt. Some non-limiting examples of such materials include neoprene, polyester, cotton, cotton polyester blends, rayon, wool, leather, fabric, linen, canvas, and/or any other suitable material.

In some cases, however, the inner material comprises polyester, a relatively breathable material, s mesh, and/or any other suitable material. Additionally, in some cases, the outer material comprises neoprene and/or any other suitable material. In this regard, some embodiments of the outer material are configured to better at insulating heat, less breathable, better at absorbing vibrations, and/or less flexible than some embodiments of the inner material. In this manner, some such embodiments of the outer material are configured to direct heat, vibrations, and/or pressure into (as opposed to away from) the user's abdomen, and the inner material is configured to help (or at least do little to impede) the belt from providing heat, vibrations, and/or pressure to the user's abdomen.

With reference now to the heating elements 30, the abdominal belt 10 can comprise any suitable type of heating elements that are configured to produce heat and that can be used with the abdominal belt. In this regards, some non-limiting examples of suitable heating elements comprise one or more graphene heating elements, tungsten heating lights, infrared heating mechanisms, heated liquid, heated wires, carbon fibers, insulated wires, and/or any other suitable type of heating element. Indeed, in some embodiments, the heating elements comprise one or more graphene heated elements.

Where the abdominal belt 10 comprises one or more heating elements 30, the heating elements can be disposed in any suitable location, including, including without limitation, in the belt 15, the abdominal pad 25, and/or in any other suitable portion of the abdominal belt. By way of non-limiting illustration, FIGS. 2A and 2D show some embodiments in which the abdominal pad 25 comprises at least some of the heating elements 30.

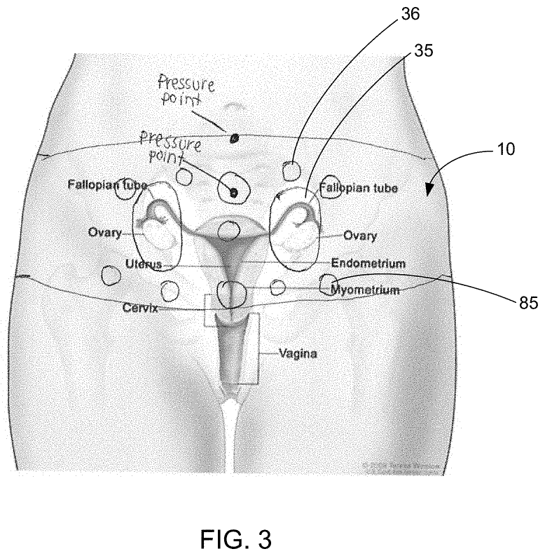

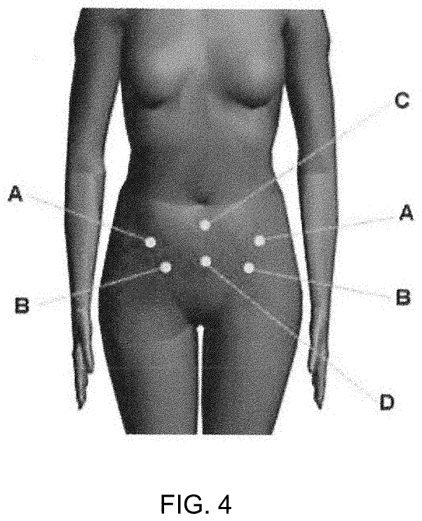

Additionally, in some embodiments, the heating elements 30 are spread across a relatively large portion of one or more parts of the abdominal belt 10 (e.g., the belt 15 and/or the abdominal pad 25). Indeed, in some embodiments, the heating elements are disposed across most, if not all of, the abdominal pad. In some other embodiments, instead of (and/or in addition to) being disposed across most if not all of the abdominal pad (and/or any other suitable portion of the abdominal belt), the heating elements are configured to provide heat in one or more specific locations.

Where the heating elements 30 are configured to provide heat (or more heat) in specific locations, the heating elements can provide heat at any portion of the abdominal belt 10, including, without limitation, the belt 15 and/or the abdominal pad 25. In some embodiments, the heating elements are configured to provide heat (or more heat than another portion of the abdominal pad or belt) at one or more specific locations (or nodes or heat nodes) on the abdominal pad, the belt, and/or any other suitable portion of the abdominal belt.

Where one or more nodes are disposed on the abdominal pad 25, such nodes can be disposed in any suitable location with respect to the abdominal pad (e.g., above, below, between, on one or more sides of, flanking, and/or in any other suitable relation to the vibrating mechanisms 35). In some embodiments, however, the nodes (or heating nodes) are disposed in locations on the abdominal pad that are configured to correspond to one or more nerve endings, veins, arteries, acupuncture points, organs, ovaries, uteruses, uterine tubes, and/or other similar trigger points that can increase blood flow, promote relaxation, ease bloating, reduce cramping, dampen pain, and/or otherwise reduce menstrual symptoms (and/or other abdominal pains) when heat is applied to such points. Indeed, in some embodiments, one or more heat nodes are configured to be disposed over a user's uterus, ovaries, uterine tubes, abdominal muscles, bowels, and/or other such trigger points when the abdominal belt is worn. By way of illustration, FIGS. 2D, 3, and 4 shows that in some embodiments, one or more heat nodes 85 are configured be disposed over (or close to) a user's uterus, uterine tubes, cervix, acupuncture pressure points, and/or in any other suitable location.

The heating nodes 85 can also be any suitable size. Indeed, in some embodiments, one or more nodes are between about 0.5 cm and about 5 cm (or any suitable subrange thereof) in width, length, and diameter (if applicable). Indeed, in some embodiments, the nodes are between about 1 cm and about 2 cm (e.g., about 1.5 cm±0.5 cm).

Where the abdominal belt 10 comprise one or more heating nodes 85, the nodes can be disposed on (or at) any suitable location of the abdominal belt, including, without limitation, on the belt 15, the abdominal pad 25, and/or any other suitable portion of the abdominal belt. Indeed, in some cases, the heat nodes are disposed between a first side and a second side of the abdominal belt (e.g., so as to not extend from either the first side or the second side of the belt. In some other embodiments, however, one or more of the heat nodes are configured to extend from the first side (e.g., inner side) and/or the second side (e.g., outer side) of a portion of the abdominal belt (e.g., the abdominal pad 25). By way of non-limiting illustration, FIG. 2D shows an embodiment in which the abdominal pad 25 comprises heat nodes 85 that extend from a second surface 90 of the abdominal pad 25.

Where the abdominal belt 10 comprises one or more heating elements 30, the heating elements can be configured to provide any desired amount of heat. Indeed, in some embodiments, the heating elements are configured to heat up to a temperature between about 32 degrees C. and about 93 degrees C. (or within any subrange thereof). Indeed, in some embodiments, the heating elements are configured to heat to a temperature between about 40 degrees C. and about 71 degrees C. (e.g., between about 45 degrees C. and about 68 degrees C.). Additionally, although some embodiments of the heating elements are configured to heat up to one preset temperature, in some other embodiments, the heating elements are configured to operate at a plurality of set temperatures and/or to have a variable temperature.

Where the abdominal belt 10 comprises one or more heating elements 30, the heating elements temperature can be controlled in any suitable manner. Indeed, in some embodiments, the abdominal belt comprises one or more temperature controls, sensors, rheostats, potentiometers, timers, programs, processors, temperature control switches, preset temperature settings, and/or any other suitable mechanisms that are configured to ensure that the heat elements are heated to one or more desired temperatures during use.

Turning now to the vibrating mechanisms 35, the described abdominal belt 10 can have any suitable type of vibrating and/or massaging mechanism that allows the abdominal belt to provide vibration and/or massage to a portion of a user's abdomen. Some examples of suitable vibrating mechanisms include, but are not limited to, one or more vibrating massagers, vibrators, bi-directional vibrators, rotary vibrators, bi-directional rotary vibrators, percussion massagers, percussion heads, hammer pads, moving knobs, kneading knobs, shiatsu massagers, massage beads, orbital massagers, and/or any other suitable type of vibrators or massagers. In some embodiments, however, the vibrating mechanisms include one or more rotary vibrational massagers.

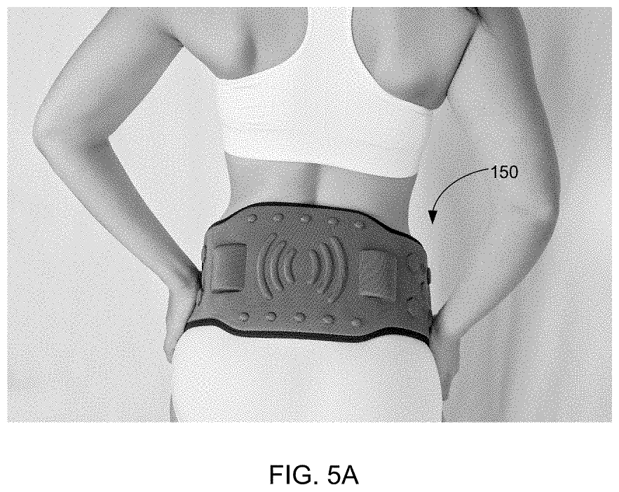

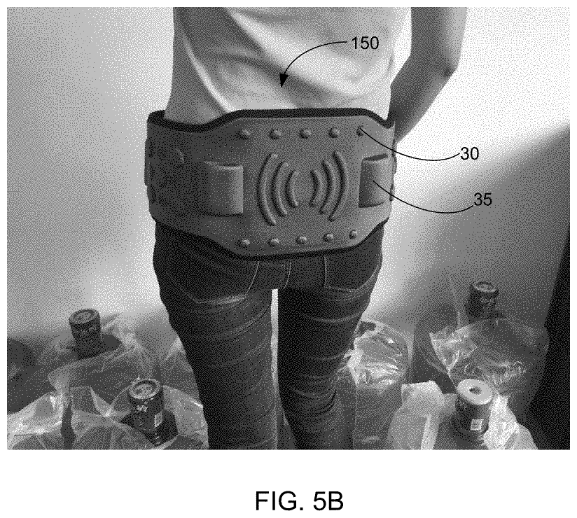

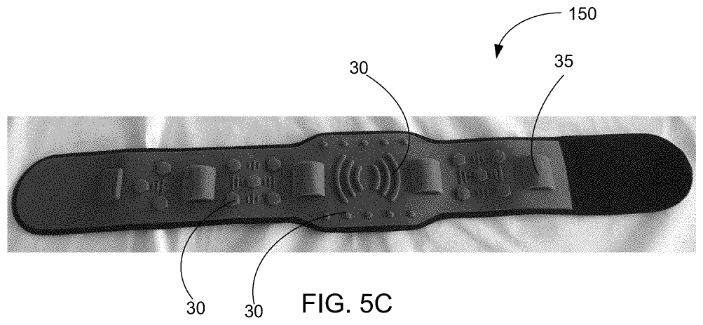

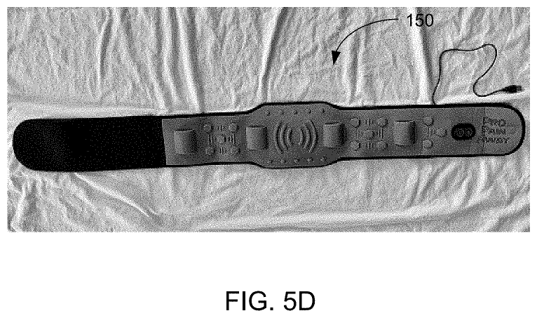

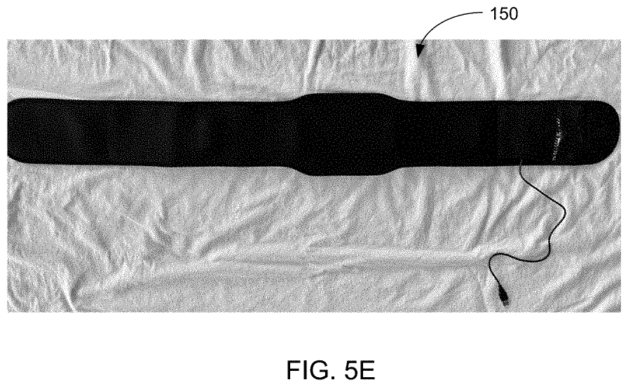

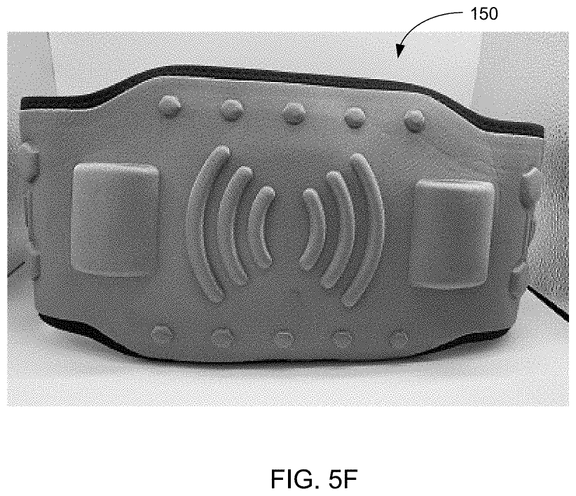

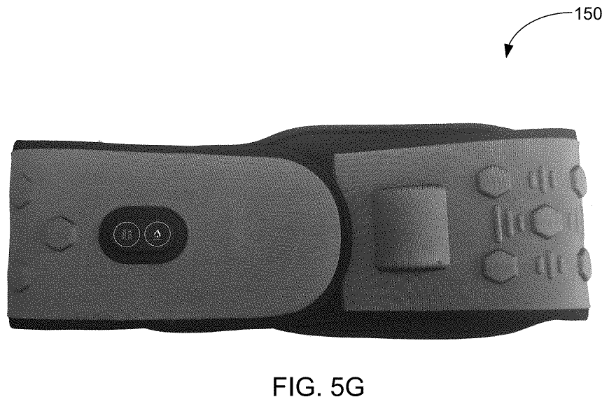

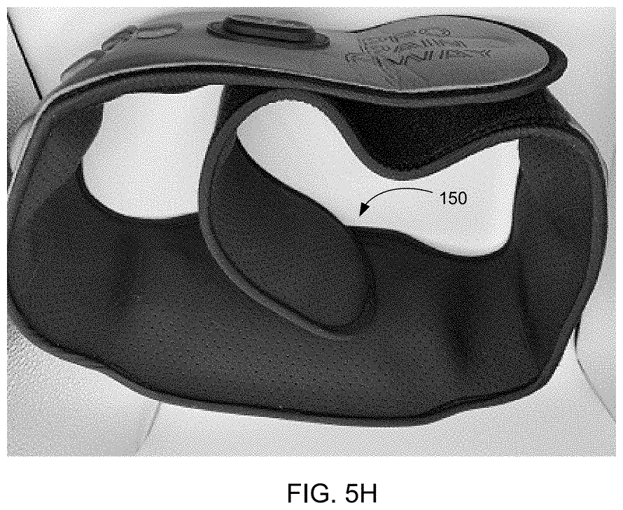

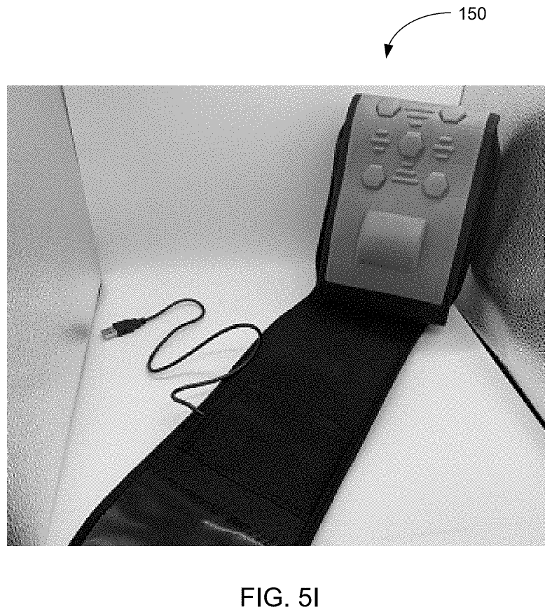

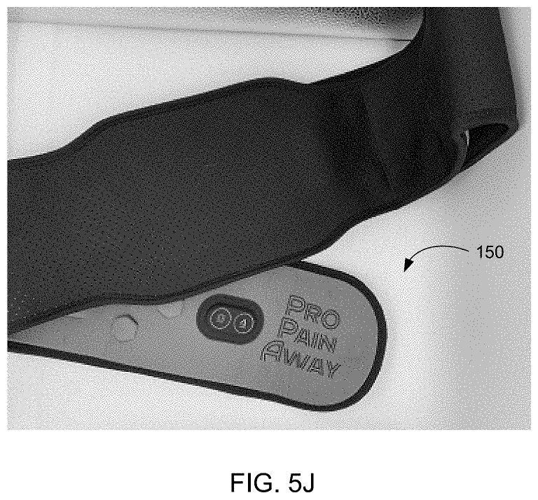

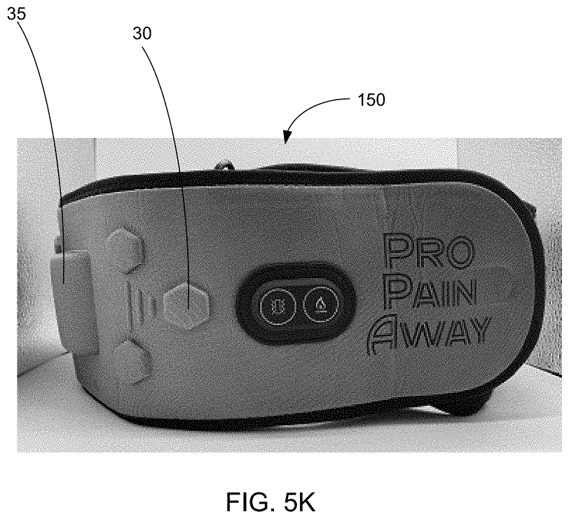

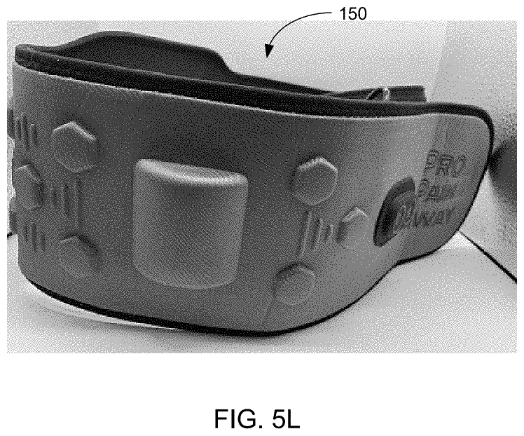

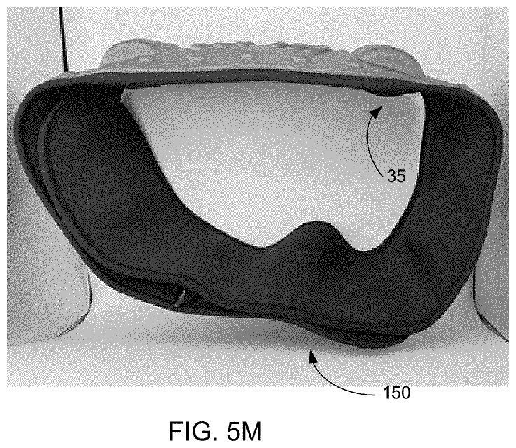

The vibrating mechanism 35 can comprise any suitable characteristic that allows it to massage or vibrate a user's abdomen. For instance, while the vibrating mechanism can vibrate at any suitable frequency, in some embodiments, it is configured to vibrate at between about 1 Hz and about 10,000 Hz (or with any subrange thereof). Indeed, in some embodiments, the vibrating mechanism comprises a high frequency vibrating mechanism that is configured to vibrate at a frequency of between about 1 Hz and about 10 Hz. Additionally, although some embodiments of the vibrating mechanism are configured to operate at only one frequency, in some other embodiments, the vibrating mechanism is configured to operate at a plurality of set frequencies and/or to have a variable frequency.

Where the vibrating mechanisms 35 comprise directional vibrators, the vibrators can vibrate in any suitable direction. Indeed, in some embodiments in which the vibrating mechanism comprises a bi-directional vibrator, the vibrator is configured to vibrate back and forth towards and away from the user, from side to side on the abdominal belt 10, up and down on the abdominal belt, and/or in any other suitable direction.

Where the abdominal belt 10 comprises one or more vibrating mechanisms 35, the mechanisms can be disposed in any suitable location, including, without limitation, at any suitable portion of the belt 15 and/or the abdominal pad 25. Indeed, in some embodiments (e.g., as shown in FIG. 5C), 1, 2, 3, 4, 5, 6, or more vibrating mechanisms 35 are disposed at any desired location of the belt. In some other embodiments (e.g., as shown in FIG. 2D), 1, 2, 3, 4, 5, 6, or more vibrating mechanisms 35 are disposed at any desired location of the abdominal pad 25. Indeed, in some embodiments, the abdominal pad comprises two or more vibrating mechanisms that are configured to be disposed near a user's ovaries (should the user have ovaries) when the user wears the abdominal belt.

In some embodiments in which the abdominal pad 25 comprises two or more vibrating mechanisms 35 that are configured to provide vibration to a user's ovary or ovaries (e.g., to increase blood flow in and/or near the ovaries, to relax the ovaries, and/or to otherwise alleviate abdominal pain), the vibrating mechanisms can be any suitable distance apart. Indeed, in some embodiments, an inner distance ID (as shown in FIG. 2D) in between (or between an innermost surface of) two vibrating mechanisms 35 is between about 2.5 cm and about 25 cm (or within any subrange thereof). In some embodiments, at least two of the vibrating mechanisms are separated by an inner distance between about 3 cm and about 10 cm. In some embodiments, the vibrating mechanisms are separated by inner distance of about 5 cm±1.5 cm. Similarly, in some embodiments, an outer distance OD (as shown in FIG. 2D) between an outermost surface of the two vibrating mechanisms is between about 6 cm and about 30 cm (or within any subrange thereof). For instance, some embodiments of the vibrating mechanisms are separated by an outer distance of between about 10 cm and about 20 cm (e.g., about 15 cm±2 cm).

The vibrating mechanisms 35 can be disposed at any suitable location with respect to the abdominal belt 10 (e.g., the abdominal pad 25, the belt 15, and/or any other suitable portion of the abdominal belt) that allows them to provide vibration to a user (e.g., to a user's abdomen). In some embodiments, one or more of the vibrating mechanisms are disposed below, above, and/or across a horizontal medial line than runs across a length AL of the abdominal pad 25. By way of non-limiting illustration, FIG. 2D shows an embodiment in which two vibrating mechanisms 35 are disposed at substantially the same height (e.g., along the same horizontal plane) on the abdominal pad 25.

In some embodiments, one or more of the vibrating mechanisms 35 are disposed between a front face (e.g., a first side, an inner side, or a portion that faces the user when worn) of the abdominal belt 10 and a back face of the abdominal belt (e.g., a second side, an outside, or a portion that faces away from the user when worn) such that the vibrating mechanisms do not protrude from the front or the back face of the abdominal belt. In some other embodiments, however, a portion of one or more of the vibrating mechanisms extend or protrude from a front face and/or a back face of a portion of abdominal belt (e.g., the abdominal pad). Indeed, in some embodiments (as shown in the FIG. 2G), the vibrating mechanisms 35 are configured to protrude from at least the front face of the abdominal pad 25 such that when the user wears the abdominal belt with the abdominal pad over the user's abdomen, one or more of the vibrating mechanisms press into the user's abdomen (e.g., further than does a portion of the abdominal pad that surrounds the vibrating mechanisms). In such a manner, the described vibrating mechanisms can be configured to effectively provide vibrations and messaging motions to a user's abdomen (e.g., ovaries, uterine tubes, etc.).

With reference to the power supply 40, the abdominal belt 10 can be powered in any suitable manner, including, without limitation, via one or more rechargeable batteries, disposable batteries, plugs, adaptors, solar cells, electrical mains, USB cords, chords, and/or in any other suitable manner. In some embodiments, the power supply comprises one or more rechargeable batteries that are coupled to the abdominal belt via one or more USB cords. In some such embodiments, the batteries are further coupled to the abdominal belt via one or more pockets, snaps, clips, mechanical connectors, and/or in any other suitable manner. By way of non-limiting illustration, FIG. 2J shows an embodiment in which the abdominal belt 10 is configured to couple a battery pack (not shown) to the abdominal belt via a USB cord 155 and a pocket 160.

Turning now to the controls 45, the abdominal belt 10 can comprise any suitable controls that allow the heating elements 30, the vibrating mechanisms 35, and/or one or more functions of the abdominal belt to turned on, turned off, adjusted, recorded, and/or otherwise be controlled. Indeed, in some embodiments, the abdominal belt comprises one or more buttons, switches, timers, apps, remote controls, and/or other mechanisms that are configured to control electronic components of the abdominal belt. By way of non-limiting illustration, FIGS. 2J and 2K show some embodiments in which the abdominal belt 10 comprise a first switch 46 to control the vibrating mechanisms 35 and a second switch 47 to control the heating elements 30. In this regard, such switches can function in any suitable manner, including, without limitation, to turn on, to turn off, to move between a variety to preset levels, and/or to otherwise control the heating elements and/or the vibrating elements. In some other embodiments, however, the described belt is configured to communicate with a processor (e.g., via wireless communications, wired communications, and/or in any other suitable manner). Thus, in some embodiments, a user is able to set programs to vary temperature and/or vibrations and/or to otherwise control the abdominal belt from the user's phone or via any other suitable device.

In addition to the aforementioned components, the described system 10 can have any other suitable component that allows it to function as intended. In one example, some embodiments of the abdominal belt optionally comprise a back pad that is configured to be disposed over a portion of a user's back so as to provide heat, vibration, and/or pressure to the lower back (e.g., at the same time and/or at different times than the abdominal pad applies heat, vibration, and/or pressure to the user's abdomen). In some such embodiments, the position of the back pad and/or the abdominal pad are fixed on the abdominal belt. In some other embodiments, however, the back pad and/or the abdominal pad are configured to be adjustable along a length of the abdominal belt (e.g., via one or more sliding mechanisms, loops through which the belt 15 runs, coupling mechanisms, hook and look fasteners, and/or any other suitable mechanism). In some such embodiments, the abdominal belt can easily be adjusted for users of different sizes.

Where the abdominal belt 10 comprises a back pad, the back pad can have any suitable component or characteristic, including, without limitation, one or more heating elements 30, vibrating mechanisms 35, controls 45, power supplies 40, and/or other components as described herein with respect to the abdominal belt 10.

In some embodiments, instead of being an abdominal belt 10, the described systems and methods include a back belt that comprises one or more of the components discussed herein with respect to the abdominal belt. In some such embodiments, however, the back belt can differ from the abdominal belt in any suitable manner, including, without limitation, by having the back pad be larger than the abdominal pad 25, by having the vibrating mechanisms be further apart from each other (or otherwise spaced and positioned differently than those of the abdominal pad), by having the back pad be more rigid than the abdominal pad, and/or in any other suitable manner. By way of non-limiting illustration, FIGS. 5A-5M show some embodiments of a back belt comprising one or more of the components discussed herein.

As another example of a suitable modification, in addition to or in place of the described coupling mechanism 20, the abdominal belt is configured to selectively increase and/or decrease pressure that the abdominal belt 10 applies to the user. Although in some embodiments, the abdominal belt 10 is tightened and/or loosened manually, in some other embodiments, the abdominal belt comprises one or more: bladders that are configured to inflate to increase pressure on the user and/or to deflate to reduce pressure on the user, automatic tightening and/or loosening mechanisms that are configured to shorten and/or extend a circumference of the abdominal belt, and/or any other suitable mechanism that is configured to automatically vary a pressure that is applied to the user by the abdominal belt.

As another example of a suitable modification, some embodiments of the abdominal belt 10 comprise one or more protuberances that are configured to extend from an inner side of the belt so as to contact one or more acupuncture spots, nerve endings, and/or other trigger spots.

As still another example of a suitable modification, in some embodiments the abdominal belt 10 is controlled by one or more programs that vary control and otherwise determine how the heating elements 30 and/or vibrating mechanisms 35 function.

As still another example of a modification, some embodiments of the described abdominal belt 10 comprise one or more transcutaneous electrical nerve stimulation units (or TENS units) that are configured to provide electrical stimulation to a user. In this regard, the belt can comprise any suitable number of probes that are configured to contact the user, including, without limitation, 1, 2, 3, 4, 5, 6, 7, 8, 9, 10, or more. Moreover, although in some embodiments, one or more of the probes of a TENS unit are relatively flush with a front face of a portion of the abdominal belt (e.g., the belt 15, the abdominal pad 25, and/or any other suitable portion of the abdominal belt), in some embodiments, one or more probes comprise one or more wires that allow the probes to extend from any suitable portion of the abdominal belt.

As yet another example of suitable modifications, in some embodiments, the abdominal belt 10 comprises one or more cooling mechanisms (e.g., allowing the belt to: switch between heating and cooling a user, chill a portion of a user, to heat one portion of the user while cooling another portion).

In still another example, some embodiments of the abdominal belt 10 are configured to produce steam and/or to diffuse aromas.

In even another example, some embodiments of the abdominal belt 10 comprise one or more magnets; pockets; shoulder straps; suspenders; straps that are configured to extend from a first portion of the belt, down below the user's crotch, and back up to a second portion of the belt; and/or any other suitable component.

The described abdominal belt 10 (and its various components) can be made in any suitable manner. In this regard, some non-limiting examples of methods for making the described abdominal belt include, cutting, molding, pressing, sewing, folding, stamping, punching, bending, shaping, extruding, connecting various pieces with one or more stitches, adhesives, mechanical fasteners (e. g. , buttons, snaps, grommets, rivets, crimps, pins, brads, staples, pegs, clips, threaded attachments, couplers, and/or any other suitable fasteners), 3D printing, additive manufacturing, welding (or melting) pieces together, and/or any other suitable method that allows the described abdominal belt to perform its intended functions. The described abdominal belt 10 can be used in any suitable manner. Indeed, in some embodiments, a user places the belt about the user's waist and/or torso, such that the abdominal pad 25 is disposed over a portion of the user's abdomen (e.g., the user's lower abdomen). In some cases, the user can then tighten the abdominal belt (e.g., via the coupling mechanism 20) to provide any suitable amount of pressure. With that the user can apply heat and vibrations to the user's abdomen in any suitable manner and for any suitable amount of time.

### Representative Operating Environment

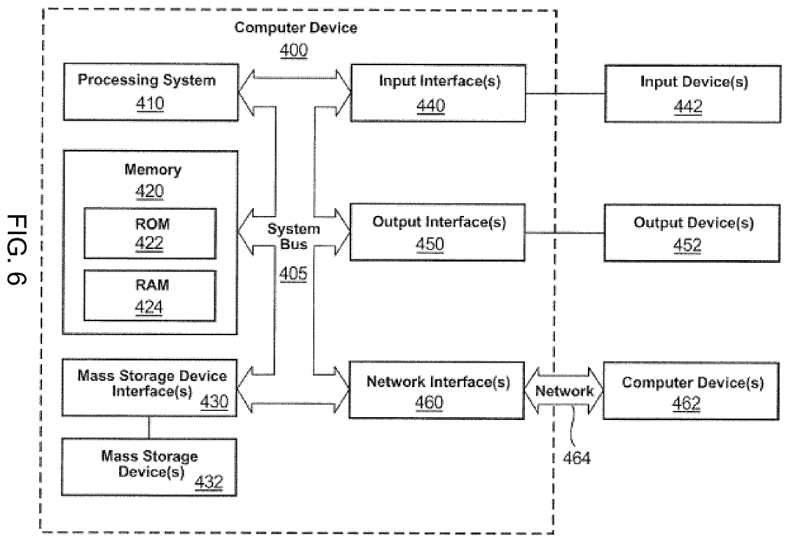

Some embodiments of the described abdominal belt 10 can be used with or in any suitable operating environment and/or software. In this regard, FIG. 6 and the corresponding discussion are intended to provide a general description of a suitable operating environment in accordance with some embodiments of the described systems and methods. As will be further discussed below, some embodiments embrace the use of one or more processing (including, without limitation, micro-processing) units in a variety of customizable enterprise configurations, including in a networked configuration, which may also include any suitable cloud-based service, such as a platform as a service or software as a service.

Some embodiments of the described systems and methods embrace one or more computer readable media, wherein each medium may be configured to include or includes thereon data or computer executable instructions for manipulating data. The computer executable instructions include data structures, objects, programs, routines, and/or other program modules that may be accessed by one or more processors, such as one associated with a general-purpose processing unit capable of performing various different functions or one associated with a special-purpose processing unit capable of performing a limited number of functions. In this regard, in some embodiments, the processing unit (e.g., a processing unit controlling the heating elements 30 and/or vibrating mechanisms 35, as mentioned above) comprises a specialized processing unit that is configured for use with the described abdominal belt 10.

Computer executable instructions cause the one or more processors of the enterprise to perform a particular function or group of functions and are examples of program code means for implementing steps for methods of processing. Furthermore, a particular sequence of the executable instructions provides an example of corresponding acts that may be used to implement such steps.

Examples of computer readable media (including, without limitation, non-transitory computer readable media) include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing unit.

With reference to FIG. 6, a representative system includes computer device 400 (e.g., an abdominal belt controlling device and/or other unit), which may be a general-purpose or special-purpose computer (or processing unit). For example, computer device 400 may be a personal computer, a notebook computer, a PDA or other hand-held device, a workstation, a minicomputer, a mainframe, a supercomputer, a multi-processor system, a network computer, a processor-based consumer device, a cellular phone, a tablet computer, a smart phone, a feature phone, a smart appliance or device, a processor, a control system, and/or any other suitable device comprising a processor.

In accordance with some embodiments, computer device 400 includes system bus 405, which may be configured to connect various components thereof and enables data to be exchanged between two or more components. System bus 405 may include one of a variety of bus structures including a memory bus or memory controller, a peripheral bus, or a local bus that uses any of a variety of bus architectures. Typical components connected by system bus 405 include processing system 410 and memory 420. Other components may include one or more mass storage device interfaces 430, input interfaces 440, output interfaces 450, and/or network interfaces 460, each of which will be discussed below.

Processing system 410 includes one or more processors, such as a central processor and optionally one or more other processors designed to perform a particular function or task. It is typically processing system 410 that executes the instructions provided on computer readable media, such as on the memory 420, a magnetic hard disk, a removable magnetic disk, a magnetic cassette, an optical disk, or from a communication connection, which may also be viewed as a computer readable medium.

Memory 420 includes one or more computer readable media (including, without limitation, non-transitory computer readable media) that may be configured to include or includes thereon data or instructions for manipulating data, and may be accessed by processing system 410 through system bus 405. Memory 420 may include, for example, ROM 422, used to permanently store information, and/or RAM 424, used to temporarily store information. ROM 422 may include a basic input/output system (“BIOS”) having one or more routines that are used to establish communication, such as during start-up of computer device 400. RAM 424 may include one or more program modules, such as one or more operating systems, application programs, and/or program data.

One or more mass storage device interfaces 430 may be used to connect one or more mass storage devices 432 to the system bus 405. The mass storage devices 432 may be incorporated into or may be peripheral to the computer device 400 and allow the computer device 400 to retain large amounts of data. Optionally, one or more of the mass storage devices 432 may be removable from computer device 400. Examples of mass storage devices include hard disk drives, magnetic disk drives, tape drives, solid state mass storage, and optical disk drives.

Examples of solid state mass storage include flash cards and memory sticks. A mass storage device 432 may read from and/or write to a magnetic hard disk, a removable magnetic disk, a magnetic cassette, an optical disk, or another computer readable medium. Mass storage devices 432 and their corresponding computer readable media provide nonvolatile storage of data and/or executable instructions that may include one or more program modules, such as an operating system, one or more application programs, other program modules, or program data. Such executable instructions are examples of program code means for implementing steps for methods disclosed herein.

One or more input interfaces 440 may be employed to enable a user to enter data (e.g., initial information) and/or instructions to computer device 400 through one or more corresponding input devices 442. Examples of such input devices include a keyboard and/or alternate input devices, such as a smart phone, a remote control, a sensor, a pin pad, a touch screen, mouse, trackball, light pen, stylus, or other pointing device, a microphone, and/or other input devices. Similarly, examples of input interfaces 440 that may be used to connect the input devices 442 to the system bus 405 include a serial port, a parallel port, a game port, a universal serial bus (“USB”), a firewire (IEEE 1394), a wireless receiver, a video adapter, an audio adapter, a parallel port, a wireless transmitter, or another interface.

One or more output interfaces 450 may be employed to connect one or more corresponding output devices 452 to system bus 405. Examples of output devices include a monitor or display screen, a speaker, a wireless transmitter, a smartphone, and the like. A particular output device 452 may be integrated with or peripheral to computer device 400. Examples of output interfaces include a video adapter, an audio adapter, a parallel port, and the like.

One or more network interfaces 460 enable computer device 400 to exchange information with one or more local or remote computer devices, illustrated as computer devices 462, via a network 464 that may include one or more hardwired and/or wireless links. Examples of the network interfaces include a network adapter for connection to a local area network (“LAN”) or a modem, BLUETOOTH, WiFi, a cellular connection, a wireless link, or another adapter for connection to a wide area network (“WAN”), such as the Internet. The network interface 460 may be incorporated with or be peripheral to computer device 400.

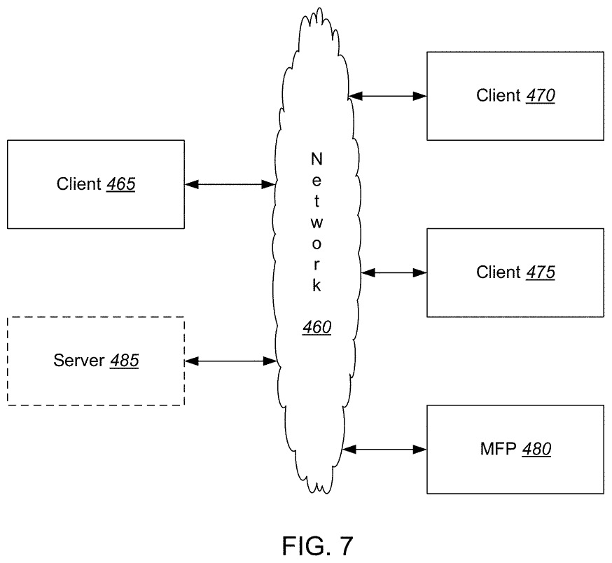

In a networked system, accessible program modules or portions thereof may be stored in a remote memory storage device. Furthermore, in a networked system computer device 400 may participate in a distributed computing environment, where functions or tasks are performed by a plurality networked computer devices. While those skilled in the art will appreciate that the described systems and methods may be practiced in networked computing environments with many types of computer system configurations, FIG. 7 represents an embodiment of a portion of the described systems in a networked environment that includes clients (465, 470, 475, etc.) connected to a server 485 via a network 460. While FIG. 7 illustrates an embodiment that includes 3 clients (e.g., abdominal belts 10, smartphones that communicate with one or more abdominal belts, etc.) connected to the network, alternative embodiments include at least one client connected to a network or many clients connected to a network. Moreover, embodiments in accordance with the described systems and methods also include a multitude of clients throughout the world connected to a network, where the network is a wide area network, such as the Internet. Accordingly, in some embodiments, the described systems and methods can allow for remote: monitoring, updating, observation, control, adjustment, trouble shooting, data collecting, recording, tracking of abdominal belt usage, system optimization, user interaction, and/or other controlling of the described abdominal belt 10 from one or more places throughout the world.

Thus, some embodiments of the current invention relate to menstrual cramps, premenstrual syndrome (PMS), ovarian swelling, and abdominal pain. More particularly, some implementations of the described invention relate to systems and methods for applying heat, vibration, and/or pressure to a lower abdominal portion of a user's body in an effort to help alleviate pain and discomfort associated with menstrual cramping, PMS, ovarian swelling, menopause, and/or a variety of other abdominal pains. While the described systems can include any suitable component, in some cases, they include a belt having a first end, a second end, and an abdominal pad that is disposed at the second end of the belt. In some cases, the abdominal pad includes one or more heating elements and/or vibrating mechanisms. Indeed, in some cases, the pad includes two vibrating mechanisms, which are spaced apart and otherwise configured to be disposed over a user's ovaries when the belt is worn. In some cases, the abdominal pad also defines an opening through which the first end of the belt is configured to extend, and the belt includes a coupling mechanism that is configured to couple the first end of the belt to a middle portion of the belt when the belt is extended through the opening and when the first end of the belt is folded back on to the middle portion of the belt.

The present invention may be embodied in other specific forms without departing from its spirit or essential characteristics. The described embodiments, examples, and illustrations are to be considered in all respects only as illustrative and not restrictive. The scope of the invention is, therefore, indicated by the appended claims rather than by the foregoing description. Each of the various elements of the described embodiments, implementations, figures, and examples can be mixed and matched with each other in any suitable manner. All changes that come within the meaning and range of equivalency of the claims are to be embraced within their scope. In addition, as the terms on, disposed on, attached to, connected to, coupled to, etc. are used herein, one object (e.g., a material, element, structure, member, etc.) can be on, disposed on, attached to, connected to, or coupled to another object—regardless of whether the one object is directly on, attached, connected, or coupled to the other object, or whether there are one or more intervening objects between the one object and the other object. Also, directions (e.g., front back, on top of, below, above, top, bottom, side, up, down, under, over, upper, lower, lateral, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

What is claimed is:

1. A menstrual belt comprising:
a belt having a first end and a second end;
an abdominal pad comprising a first vibrating mechanism and a heating element,
wherein a first end portion of the abdominal pad is disposed at the second end of the belt,
wherein a second end portion of the abdominal pad defines an opening through which the first end of the belt is configured to extend, and
wherein the belt further comprises a coupling mechanism that is configured to couple the first end of the belt to a middle portion of the belt when the first end of the belt is extended through the opening and the first end of the belt is folded back on the middle portion of the belt.

2. The belt of claim 1, wherein the abdominal pad further comprises a second vibrating mechanism.

3. The belt of claim 2, wherein the first and second vibrating mechanisms are separated from each other on the abdominal pad by less than about 12 cm.

4. The belt of claim 1, wherein the abdominal pad comprise an inner surface that is configured to face a user when the user wears the menstrual belt, wherein the abdominal pad comprises an outer surface that is disposed substantially opposite to the inner surface, wherein the heating element is disposed between the inner surface and the outer surface, and wherein the outer surface is more heat insulative than is the inner surface.

5. The belt of claim 4, wherein the outer surface comprises neoprene.

6. The belt of claim 1, wherein the heating element is disposed at a first node, the first node being less than about 3 cm in both length and diameter.

7. The belt of claim 1, wherein the first vibrating mechanism is configured to protrude from a first side of the abdominal pad, such that the first vibrating mechanism is configured to press into an abdominal portion of a user when the user wears the abdominal belt over the abdominal portion of the user and the belt is tightened onto the user.

8. The belt of claim 1, wherein the abdominal portion is less than about 36 cm in length and less than about 20 cm in height.

9. The belt of claim 8, wherein a height of the of the abdominal portion is between about 2 cm and about 6 cm greater than a height of the belt.

10. The belt of claim 1, wherein the opening comprises an elongated grommet and wherein the coupling mechanism comprises a hook and loop fastener.

11. A menstrual belt comprising:
a belt having a first end and a second end;
an abdominal pad comprising a first vibrating mechanism, a second vibrating element, and a first heating element,
wherein a first end portion of the abdominal pad is coupled to the second end of the belt, and
wherein the first and second vibrating mechanisms are separated from each other on the abdominal pad by less than about 10 cm.

12. The belt of claim 11, wherein a second end portion of the abdominal pad defines an opening through which the first end of the belt is configured to extend, and wherein the menstrual belt further comprises a coupling mechanism that is configured to couple the first end of the belt to a middle portion of the belt when the first end of the belt is extended through the opening and the belt is folded back on itself.

13. The belt of claim 11, wherein the abdominal pad comprise an inner surface that is configured to face a user when the user wears the menstrual belt, wherein the abdominal pad comprises an outer surface that is disposed substantially opposite to the inner surface, wherein the first heating element is disposed between the inner surface and the outer surface, and wherein the outer surface is more heat insulative than is the inner surface.

14. The belt of claim 13, wherein the first heating element is disposed in a node that extends from the outer surface of the abdominal pad.

15. The belt of claim 11, wherein the first vibrating mechanism extends from both a first side and a second side of the abdominal pad.

16. The belt of claim 11, wherein the abdominal pad comprise an inner surface that is configured to face a user when the user wears the menstrual belt, wherein the abdominal pad comprises an outer surface that is disposed substantially opposite to the inner surface, wherein the first heating element is disposed between the inner surface and the outer surface, wherein the outer surface is more heat insulative than is the inner surface, and wherein the first vibrating mechanism extends from both a first side and a second side of the abdominal pad.

17. A method for applying at least one of heat, vibration, and pressure to an abdominal portion of a user, the method comprising:
obtaining a belt having:
a first end;
a second end; and
an abdominal pad comprising a first vibrating mechanism, a second vibrating element, and a first heating element,
wherein a first end portion of the abdominal pad is coupled to the second end of the belt, and
wherein the first and second vibrating mechanisms are separated from each other on the abdominal pad by less than about 12 cm; and
coupling the belt on the user such that the abdominal pad is disposed over a lower abdomen of the user and such that the first and second vibrating mechanisms are respectively configured to be disposed adjacent to a first and a second ovary of the user.

18. The method of claim 17, wherein a second end portion of the abdominal pad defines an opening through which the first end of the belt is configured to extend, wherein the belt further comprises a coupling mechanism that is configured to couple the first end of the belt to a middle portion of the belt when the first end of the belt is extended through the opening and the belt is folded back on itself, wherein the coupling mechanism comprises a hook and loop fastener, and wherein the method further comprises tightening the belt on the user and coupling the first end of the belt to the middle portion of the belt.

19. The method of claim 17, wherein the abdominal pad comprise an inner surface that is configured to face a user when the user wears the belt, wherein the abdominal pad comprises an outer surface that is disposed substantially opposite to the inner surface, wherein the first heating element is disposed between the inner surface and the outer surface, and wherein the outer surface is more heat insulative than is the inner surface.

20. The method claim 17, wherein the first and second vibrating mechanisms are configured to extend from an inner side of the abdominal pad, and wherein the method comprises wearing the belt such that the first and second vibrating mechanisms press into the lower abdomen of the user.
